(19) United States
(12) Patent Application Publication
(10) Pub. No.: US 2013/0073037 A1
(43) Pub. Date: Mar. 21, 2013

(54) PROSTHETIC HEART VALVE IMPLANTATION

(71) Applicants: Peter W. Gregg, Santa Cruz, CA (US); Daniel Hildebrand, San Francisco, CA (US)

(72) Inventors: Peter W. Gregg, Santa Cruz, CA (US); Daniel Hildebrand, San Francisco, CA (US)

(73) Assignee: BOSTON SCIENTIFIC SCIMED, INC., Maple Grove, MN (US)

(21) Appl. No.: 13/553,128

(22) Filed: Jul. 19, 2012

Related U.S. Application Data
(60) Provisional application No. 61/509,791, filed on Jul. 20, 2011.

(51) Int. Cl. A61F 2/24; B23P 11/00

(52) U.S. Cl. 623/2.19; 29/428

(57) ABSTRACT

A prosthetic heart valve includes an expandable stent and a leaflet assembly coupled to the expandable stent. The expandable stent is radially expandable from a first size for intraluminal delivery through a body passageway to a second size for implantation of the prosthetic heart valve in the body passageway. The leaflet assembly includes leaflets movable between an open position permitting flow past the expanded stent and a closed position substantially restricting flow past the expanded stent. Each of the leaflets is coaptable with each of the other leaflets in the closed position to define a coaptation region of about 15 percent to about 35 percent of the overall height of each of the plurality of leaflets.

### CROSS-REFERENCE TO RELATED APPLICATION

This application claims priority under 35 USC §119(e) to U.S. Provisional Patent Application Ser. No. 61/509,791, filed on Jul. 20, 2011, the entire contents of which are hereby incorporated by reference.

### TECHNICAL FIELD

The following disclosure relates to replacement heart valves and, more particularly, to replacement heart valves including leaflets.

### BACKGROUND

Heart valve surgery can be used to repair or replace diseased heart valves. For example, heart valve replacement may be indicated when there is a narrowing of the native heart valve, commonly referred to as stenosis, or when the native valve leaks or regurgitates. Surgery to repair or replace diseased heart valves can be an open-heart procedure, conducted under general anesthesia, in which an incision is made through the patient's sternum (sternotomy), and the patient's heart is stopped while blood flow is rerouted through a heart-lung bypass machine.

Post-surgery, patients temporarily may be confused due to emboli and other factors associated with the heart-lung machine. The first 2-3 days following surgery are spent in an intensive care unit where heart functions can be closely monitored. The average hospital stay is between 1 to 2 weeks, with several more weeks to months required for complete recovery. Given its highly invasive nature, this type of surgery is often unavailable as a treatment option for patients with compromised ability to recover.

### SUMMARY

A prosthetic heart valve replaces the function of a native heart valve such that the prosthetic valve regulates the flow of blood through the heart.

In one aspect, a prosthetic heart valve includes a radially expandable stent and a leaflet assembly coupled to the expandable stent. The stent is radially expandable from a first size for intraluminal delivery through a body passageway to a second size for implantation of the prosthetic heart valve in the body passageway. The leaflet assembly includes a plurality of leaflets movable between an open position permitting flow past the expanded stent and a closed position substantially restricting flow past the expanded stent. The plurality of leaflets are coaptable with one another in the closed position to define a coaptation region of about 15 percent to about 35 percent of the overall height of each of the plurality of leaflets.

In some embodiments, each of the plurality of leaflets has an overall height of about 19 mm and the coaptation region is about 3 mm to about 6 mm.

In certain embodiments, at least a portion of each leaflet of the plurality of leaflets is sutured to at least a portion of each of the other leaflets of the plurality of leaflets.

In some embodiments, the leaflet assembly further includes a plurality of posts secured to the stent, wherein at least a portion of each leaflet is sutured to at least one post. The stent can be tubular and a longitudinal axis of each post can be substantially parallel to a longitudinal axis of the stent. For example, the plurality of posts can be substantially evenly spaced about an interior surface of the tubular stent.

In certain embodiments, each leaflet of each of the plurality of leaflets has a first end portion, a second end portion, and a belly portion extending therebetween. The first end portion of each leaflet can be secured to the stent and the second end portion of each leaflet can be movable relative to the respective second end portions of each of the other leaflets as the leaflets move from the closed position to the open position. Each leaflet can be sized such that the belly portion of each respective leaflet is spaced from the stent.

In some embodiments, each leaflet is deflectable about a first axis defined by the leaflet and the respective deflections of the plurality of leaflets about the respective first axes under gravity varies from one another by less than about 0.125 inches. Additionally or alternatively, each leaflet is deflectable about a second axis defined by the leaflet and the respective deflections of the plurality of leaflets about the respective second axes under gravity varies from one another by less than about 0.125 inches. The first axis can be, for example, substantially perpendicular to the second axis.

In certain embodiments, the coaptation region is defined by engagement of three leaflets of the plurality of leaflets.

In some embodiments, the stent is tubular and at least a portion of each of the plurality of leaflets is disposed in a volume defined by the stent. At least a portion of the coaptation region can be disposed along a center, longitudinal axis of the stent.

In certain embodiments, each of the plurality of leaflets has a thickness of between about 0.010 inches to about 0.015 inches.

In some embodiments, the plurality of leaflets include biological tissue. For example, the biological tissue can be one or more of the following: bovine pericardium, equine pericardium, and porcine pericardium.

In certain embodiments, the stent is self-expandable from at least a portion of the radial expansion from the first size to the second size.

In some embodiments, the stent is mechanically expandable from at least a portion of the radial expansion from the first size to the second size. Additionally or alternatively, the stent can have a first end portion and a second end portion, with the first end portion and the second end portion movable toward one another to radially expand the stent.

In certain embodiments, the second size of the stent is sized to secure at least a portion of the stent to the body passageway.

In some embodiments, the stent includes at least one braided wire. For example, the at least one braided wire can have an outer diameter of about 0.008 inches to about 0.020 inches.

In certain embodiments, at least a portion of the stent is deformable to define a non-circular cross-section when implanted in the body passageway.

In another aspect, a method of manufacturing a prosthetic heart valve includes forming a leaflet assembly and securing the leaflet assembly to a radially expandable stent. Forming the leaflet assembly includes engaging a plurality of leaflets with one another. The leaflet assembly is secured to the radially expandable stent such that the plurality of leaflets are movable between an open position permitting flow past the expanded stent and a closed position substantially restricting flow past the expanded stent. Each of the plurality of leaflets is coaptable with each of the other leaflets in the closed position to define a coaptation region of about 15 percent to about 35 percent of the overall height of each of the plurality of leaflets.

In some embodiments, forming the leaflet assembly further includes cutting each of the plurality of leaflets from a sheet of biological tissue. For example, cutting each leaflet can include pressing a steel die on the sheet of biological tissue. The biological tissue can be one or more of the following: bovine pericardium, equine pericardium, and porcine pericardium.

In certain embodiments, each leaflet of the leaflet assembly is moistened. For example, each leaflet of the leaflet assembly can be moistened by exposing each leaflet to a moistening solution. The moistening solution can include saline. The exposure of each leaflet to the moistening solution can be done before and after forming the leaflet assembly. Additionally or alternatively, the exposure of each leaflet to the moistening solution can be done before and after securing the leaflet subassembly to the radially expandable stent.

In some embodiments, the moistening of each leaflet of the leaflet assembly includes storing each leaflet in a moistening solution. Additionally or alternatively, moistening each leaflet of the leaflet assembly can include storing the leaflet assembly in the open position in a moistening solution.

In certain embodiments, forming the leaflet assembly includes suturing at least a portion of each leaflet to each of the other of the plurality of leaflets.

In some embodiments, securing the leaflet assembly to the expandable stent includes suturing at least a portion of each leaflet to the expandable stent. For example, the leaflet assembly can include a plurality of posts and securing the leaflet assembly to the stent can include securing each of the posts to the stent.

In certain embodiments, the plurality of leaflets are selected. For example, each leaflet defines a first axis and selecting the plurality of leaflets includes selecting leaflets that deflect about each of their respective first axes under gravity by an amount that varies from one another by less than about 0.125 inches. Additionally or alternatively, each leaflet defines a second axis and selecting the plurality of leaflets further includes selecting the leaflets that deflect about each of their respective second axes under gravity by an amount that varies from one another by less than about 0.125 inches.

Embodiments can include one or more of the following advantages.

In some embodiments, the plurality of leaflets define a coaptation region that is about 15 percent to about 35 percent of the overall height of each of the plurality of leaflets. In a prosthetic heart valve configured for intraluminal delivery to an implantation site in a body passageway, a coaptation region of this size results in redundant coaptation that can improve the robustness of leaflet coaptation when the prosthetic heart valve is implanted along a non-circular portion of a body passageway (e.g., at an implantation site that produces non-uniform stresses on the stent). For example, such redundant coaptation can prevent central leakage through the leaflets of the valve when the valve is in the closed position, particularly when the valve is implanted in an out-of-round/calcific annuli.

In certain embodiments, the prosthetic heart valve is produced by selecting leaflets that have similar flexibility characteristics (e.g., flexibility variation about one or two axes under the force of gravity that varies by less than about 0.125 inches). Matching leaflets in this way can reduce the likelihood that the leaflets will delaminate as a result of uneven coaptation, even if the prosthetic heart valve is disposed along a non-circular body passage.

In other embodiments, the prosthetic heart valve is produced by moistening the leaflets throughout the process of assembling the prosthetic heart valve and/or storing the leaflets in a moistening solution. This moistening regiment reduces the likelihood that the leaflets will begin to delaminate during the manufacturing process which can, in turn, reduce the likelihood that the leaflets will become delaminated during use. For at least this reason, moistening the leaflets during the manufacturing process can facilitate the use of a relatively large coaptation region that can allow proper coaptation under conditions in which the stent is subject to non-uniform stresses in the body passageway.

Other features, objects, and advantages will be apparent from the description and drawings, and from the claims.

### DESCRIPTION OF DRAWINGS

FIG. 1 is a partial cut-away view of a replacement valve in an unexpanded delivery configuration within a delivery system.

FIG. 2 is an isometric view of the replacement valve of FIG. 1 in an expanded state.

FIG. 3 is a top-down, plan view of the replacement valve of FIG. 1

FIG. 4 is a cross-sectional view of the replacement valve of FIG. 1, taken along the line A-A of FIG. 3.

FIG. 5 is a schematic representation of leaflet separation from a tissue sheet.

FIG. 6 is a top, plan view of a flattened leaflet of the replacement valve of FIG. 1.

FIGS. 7A and 7B are schematic representations of leaflet deflection along a first axis and a second axis.

FIGS. 8A-8C are schematic representations of the deployment of the replacement valve of FIG. 1 to replace an aortic valve.

FIG. 9 is a schematic representation an end-view of the valve of FIG. 1 in a deployed position, with the valve in a closed position.

FIG. 10 is a schematic representation of an end-view of the valve of FIG. 1 in a deployed position, with the valve in an open position.

FIG. 11 is a schematic representation of a cross-sectional side view of the valve of FIG. 1 in a deployed position, taken along the line B-B in FIG. 9.

Like reference symbols in the various drawings indicate like elements.

### DETAILED DESCRIPTION

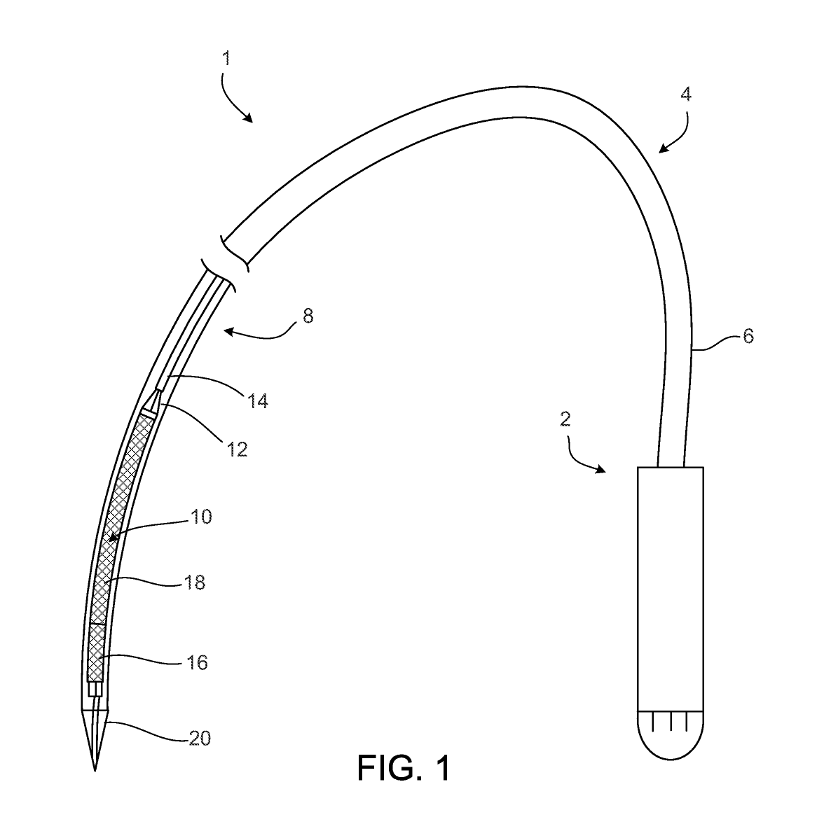

Referring to FIG. 1, a delivery system 1 includes a control handle 2, an external sheath 4, and a replacement valve 10. In the undeployed configuration shown in FIG. 1, a distal portion 8 of the external sheath 4 is disposed about the replacement valve 10 in an unexpanded state such that the replacement valve 10 can be moved through a body passageway (e.g., a femoral artery) to an implantation site (e.g., an aortic valve) with minimal invasiveness and/or trauma to the implant recipient. For example, a multi-lumen catheter 14 can be disposed within the external sheath 4 and, as described in further detail below, the replacement valve 10 can be advanced through a body passageway, to an implantation site, by moving the multi-lumen catheter 14 over a guidewire (not shown in FIG. 1) extending through the delivery system 1 from the control handle 2 to a nosecone 20 at the distal portion 8 of the external sheath 4.

Once the replacement valve 10 has been advanced to the implantation site, the control handle 2 is manipulated to move the distal portion 8 of the external sheath 4 proximally to expose the replacement valve 10 at the implantation site. As described in further detail below, the exposed replacement valve 10 can radially expand from the unexpanded state for intraluminal delivery through a body passageway to an expanded state for implantation of the replacement valve in the body passageway. In certain embodiments, the replacement valve 10 is mechanically expanded from the unexpanded state to at least a portion of the expanded state. For example, as shown in FIG. 1, actuation elements 12 can extend through the multi-lumen catheter to engage the replacement valve 10. Continuing with this example, the replacement valve 10 self-expands upon withdrawal of the distal portion 8 of the external sheath 4 and the control handle 2 moves actuation elements 12 to further expand the replacement valve 10 (e.g, by foreshortening the valve) for engagement with the body passageway at the implantation site.

Referring now to FIGS. 1-4, the replacement valve 10 includes a leaflet assembly 16 and a stent 18. The leaflet assembly 16 is coupled to the stent 18 such that the leaflet assembly 16 is disposed within the volume defined by the stent 18. Thus, for example, the leaflet assembly 16 is disposed within the stent 18 when the stent 18 is in the unexpanded state and is being moved through the body passageway. The leaflet assembly 16 is also disposed within the stent 18 when the stent is in the expanded state at the implantation site.

The stent 18 is substantially tubular and defines a volume extending from a first end portion 21 to a second end portion 22 and defines an outer diameter of the replacement valve. The substantially tubular shape of the stent 18 can be defined by 1, 2, 3, or 4 braided wires (e.g., wires each having an outer diameter of about 0.008 inches to about 0.020 inches). In some embodiments, the stent 18 is nitinol. In certain embodiments, the stent 18 has a diameter of about 20 mm to about 30 mm in an expanded, unstressed state. When the stent 18 is in the expanded state in a body passageway, the expanded stent engages the body passageway to hold the replacement valve 10 in place.

The leaflet assembly 16 is substantially symmetrically disposed about a center axis 11 defined by the stent 18 in a fully expanded, unstressed state. However, unlike valves implanted through open-heart procedures, the amount of preparation (e.g., scraping, excision of native leaflets, etc.) that can be done to the implantation site prior to the intraluminal delivery of the replacement valve 10 is limited. For at least this reason, the stent 18 can be subjected to non-uniform stresses at the implantation site. For example, the stent 18 can be subjected to non-uniform stresses (e.g., non-uniform radial stresses) as a result of being positioned along a portion of a body passageway that is non-circular, includes a calcium deposit, and/or includes fused native leaflets. As described below, the leaflet assembly 16 is sized to open and close when the stent 18 is subjected to these non-uniform stresses at the implantation site.

The leaflet assembly 16 includes three leaflets 30a, 30b, 30c and posts 26a, 26b, 26c. Each post 26a, 26b, 26c is coupled (e.g., sutured) to an interior surface of the stent 18, substantially evenly spaced about the interior surface of the stent 18. This relative positioning of the posts 26a, 26b, 26c can facilitate symmetric mounting of the leaflets 30a, 30b, 30c relative to the expanded, unstressed stent 18. Each post 26a, 26b, 26c is substantially cylindrical and coupled (e.g., sutured) to an interior surface of the stent 18 such that a longitudinal axis of each post 26a, 26b, 26c is substantially parallel to the center axis 11 of the expanded stent 18. Buckles 28a, 28b, 28c are coupled to the stent 18 along the interior surface of the stent 18 and are substantially aligned with respective posts 26a, 26b, 26c. Actuation elements 12 can draw the first and second end portions 21, 22 of the stent 18 toward one another to move the posts 26a, 26b, 26c toward buckles 28a, 28b, 28c. Additionally or alternatively, the actuation elements 12 can draw the first and second end portions 21, 22 of the stent 18 toward one another (e.g., to foreshorten the stent 18) to expand the stent 18 radially into secure engagement with the body passageway. In some embodiments, the stent 18 is radially expandable from a first size for intraluminal delivery to a second size and is further radially expandable by moving the first and second end portions 21, 22 of the stent 18 toward one another.

For the sake of clarity, the mounting of leaflet 30a is described, and it should be appreciated that the mounting of leaflets 30b and 30c is analogous to the mounting of leaflet 30a. Leaflet 30a has a first end portion 37, a second end portion 39, and a belly portion 41 extending therebetween. The first end portion 37 of the leaflet 30a is coupled to the stent 18 by stent sutures 36, for example, extending circumferentially around the second end portion 22 of the stent 18. The leaflet 30a is sutured to each of the other leaflets by leaflet sutures 32 extending generally in a direction from the first end portion 37 to the second end portion 39 of each leaflet. The second end portion 39 of leaflet 30a is coupled to two posts 26a, 26c such that the second end portion 39 of leaflet 30a is movable relative to the respective second end portions of each of the other leaflets as the leaflets 30a, 30b, 30c move from the closed position to the open position. In some embodiments, leaflet 30a is sized relative to the expanded dimension of the stent 18 such that the belly portion 41 of the leaflet 30a is spaced from the stent 18 as the leaflet 30a moves in response to changes in flow through the replacement valve 10. This relative spacing can, for example, reduce the likelihood that the leaflet 30a will wear out through repeated contact with the stent.

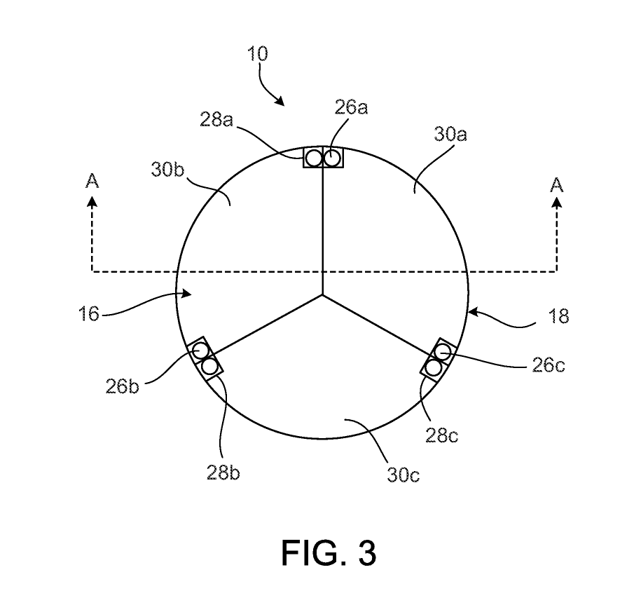

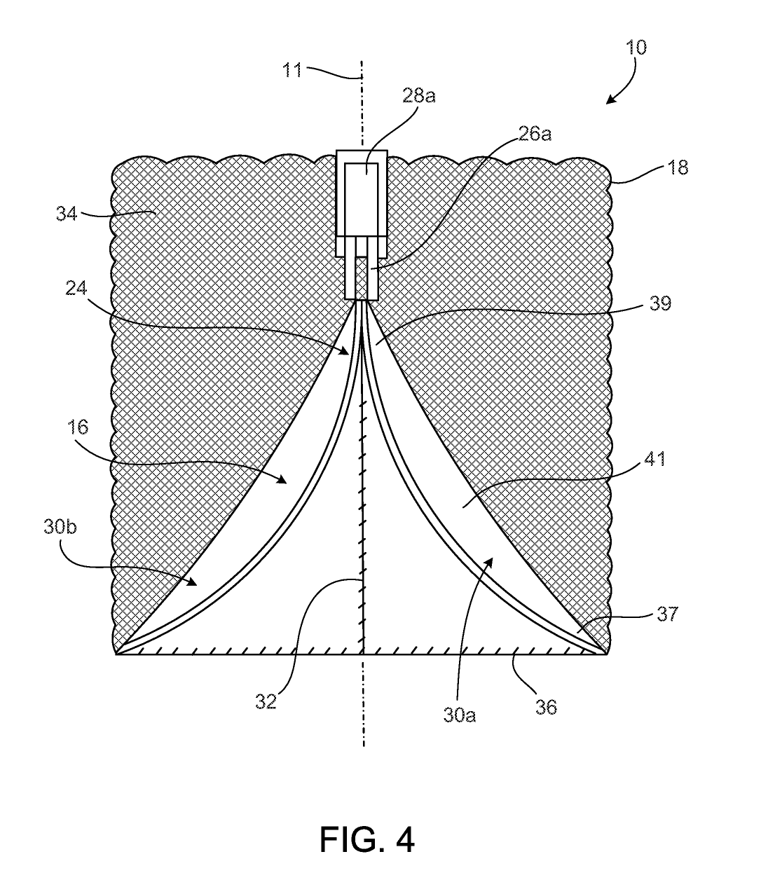

Given that leaflets 30b and 30c are mounted in a manner analogous to the mounting of leaflet 30a, the mounted leaflets 30a, 30b, 30c are movable between an open position (permitting flow past the expanded stent 18) when fluid flows from a second end portion 22 to a first end portion 21 of the expanded stent 18 and a closed position (substantially restricting flow past the expanded stent 18) when fluid flows from the first end portion 21 to the second end portion 22 of the expanded stent 18. In the closed position (shown in FIGS. 3 and 4), the leaflets 30a, 30b, 30c are coaptable with one another to define a coaptation region 24. At least a portion of the coaptation region is disposed substantially along the center axis 11 of the stent 18 when the stent 18 is in an expanded, unstressed state.

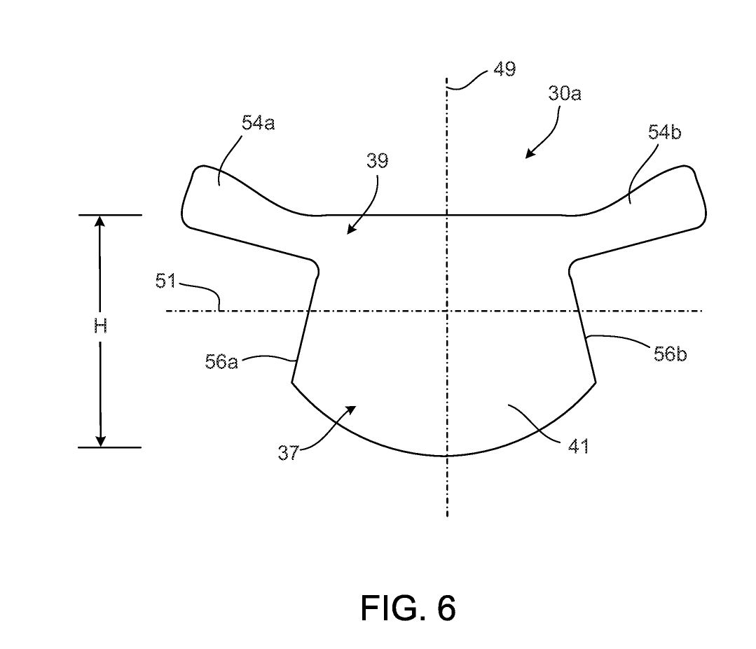

The coaptation region 24 is about 15 percent to about 35 percent (e.g., about 17 percent to about 33 percent, about 20 percent to about 30 percent, about 23 percent to about 27 percent, about 24 percent to about 25 percent) of the overall height H (FIG. 6) of each of the plurality of leaflets. For example, the overall height of each leaflet can be about 19 mm and the leaflets 30a, 30b, 30c can be supported on the expanded stent 18 such that coaptation region 24 is about 3 mm to about 6 mm (e.g., about 4 mm). In general, the amount of material associated with a coaptation region of a larger size could result in prohibitively large sheathing forces required to sheath the replacement valve 10 for intraluminal delivery. Additionally or alternatively, creating a coaptation region of a larger size could make intraluminal delivery more difficult to the extent that larger sized coaptation regions are associated with a larger amount of material that would increase the outer diameter of the delivery system 1.

As described below, the size of the coaptation region 24 improves the likelihood of proper coaptation of the leaflets 30a, 30b, 30c if the stent 18 is intraluminally delivered to an implantation site that is non-circular or an otherwise uneven portion of a body passageway. Accordingly, the coaptation region 24 is generally larger than a coaptation region of a valve implanted through open-heart procedures, which typically afford an opportunity to improve the uniformity (e.g., roundness) of the implantation site prior to implanting the valve. As also described below, leaflet wear that could otherwise result from large coaptation sizes is reduced by selecting leaflets with matching flexibility and moistening the leaflets throughout the process of making the leaflet assembly 16 and before and after attaching the leaflet assembly 16 to the stent 18.

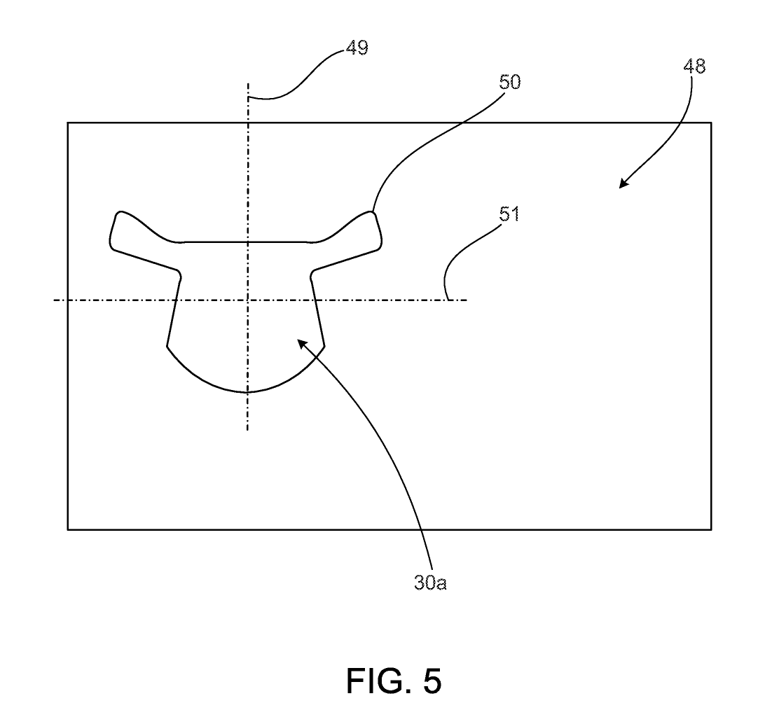

Referring now to FIG. 5, the leaflet 30a is cut from a substantially flat sheet 48 using, for example, a cutting die 50 in the shape of the leaflet 30a. The flat sheet 48 can be a biological tissue, including one or more of the following: bovine pericardium, equine pericardium, and porcine pericardium. The leaflet 30a can be cut from a portion of the flat sheet 48 having a thickness of between about 0.010 inches to about 0.015 inches such that the leaflet 30a will have a thickness in this range. The cutting die 50 can be placed on the sheet 48 (e.g., on a substantially uniform portion of the sheet 48) and pressed (e.g., using a machine press) into the sheet 48 to cut the leaflet 30a.

For clarity of explanation, the cutting of leaflet 30a is shown in FIG. 5. It should be appreciated, however, that leaflets 30a, 30b and 30c have substantially similar geometries and leaflets 30b and 30c can be cut in a manner analogous to the cutting of leaflet 30a. For example, leaflets 30b and 30c can also be cut from the flat sheet 48. As another example, leaflets 30b and 30c can be cut from different flat sheets of the same material.

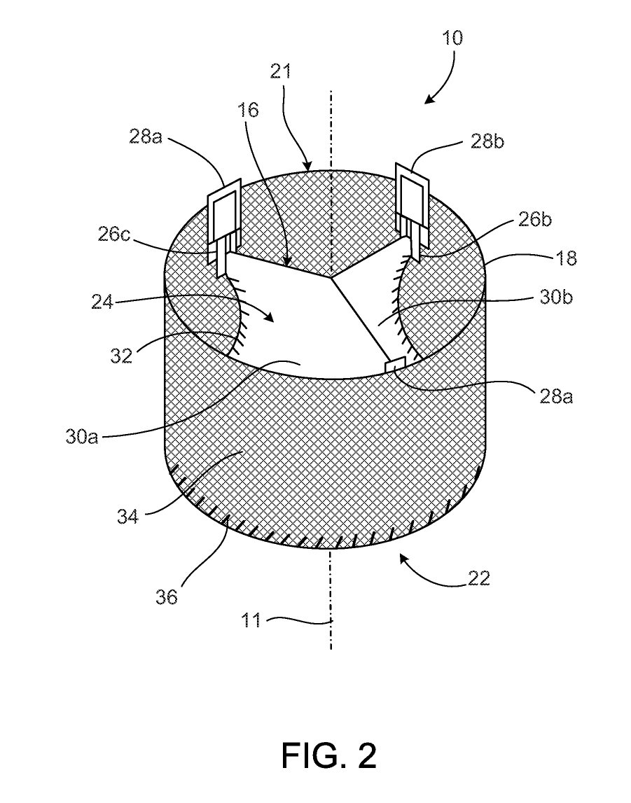

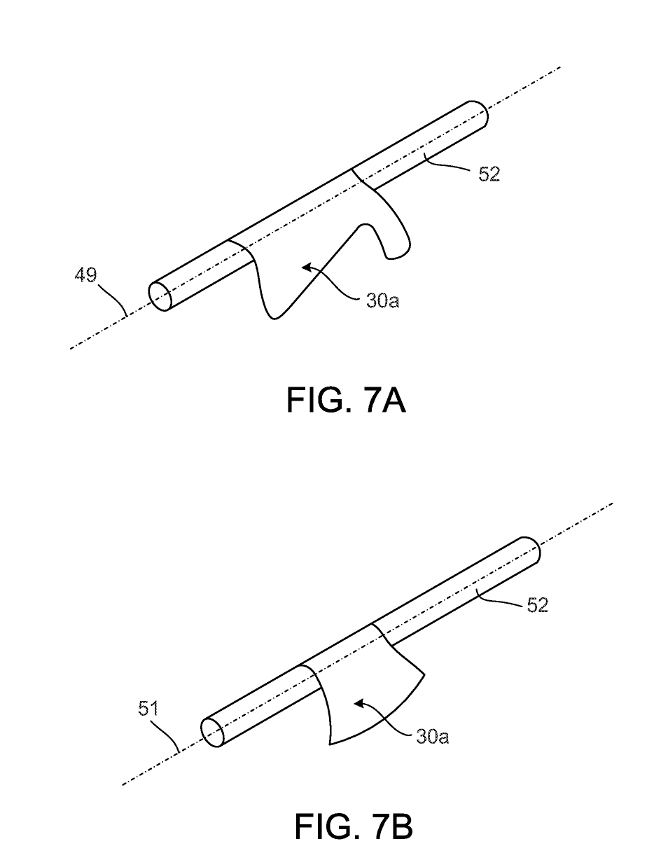

Referring now to FIGS. 6 and 7A-7B, the resulting leaflet 30a is substantially symmetrical about a first axis 49 extending through the center of mass of the leaflet 30a, in a direction extending generally from the first end portion 37 to the second end portion 39 of the leaflet 30a, through the center of mass of the leaflet 30a. For example, the leaflet 30a has tabs 54a and 54b disposed on either side of the first axis 49. Similarly, the leaflet 30a has side portions 56a and 56b disposed on either side of the first axis 49. Side portions 56a and 56b are sutured (e.g. using leaflet sutures 32 in FIGS. 2 and 4) to respective side portions of the other leaflets 30b and 30c to form a substantially tubular structure. The tabs 54a and 54b are sutured to the posts 26a and 26c (FIG. 3) to couple the leaflet 30a to the stent 18.

The leaflet 30a also defines a second axis 51 substantially perpendicular to the first axis 49 and extending through the center of mass of the leaflet 30a. The first end portion 37 is disposed on one side of the second axis 51 and the second end portion is disposed on the other side of the second axis 51.

The leaflet 30a can be suspended about a support 52 substantially parallel to the first axis 49 (FIG. 7A), and the deflection of the leaflet 30a about the first axis 49 under the force of gravity can be measured. Similarly, the leaflet 30a can be suspended about the support 52 substantially parallel to the second axis 51 (FIG. 7B), and the deflection of the leaflet 30a about the second axis 51 under the force of gravity can also be measured. In some embodiments, the leaflet 30a exhibits asymmetrical biaxial deflection, with the same load resulting in a different amount of deflection about the first axis 49 than about second axis 51. In some embodiments, the deflection about the second axis 51 can be greater than the deflection about the first axis 49 for a given load (e.g., under the force of gravity). For the sake of clarity, the deflection of leaflet 30a has been discussed. However, it should again be appreciated that leaflets 30b and 30c have geometries similar to the geometry of leaflet 30a and will be deflected about the support 52 in an analogous manner to quantify the deflection characteristics of these leaflets under gravity.

Given the relatively large size of the coaptation region 24 (FIG. 4), flexibility of the leaflets 30a, 30b, and 30c are matched to reduce the likelihood of mismatched contact between two or more of the leaflets 30a, 30b, and 30c that can, in certain instances, result in delamination. In some embodiments, the leaflets 30a, 30b, and 30c are selected to have similar deflections about the first axis 49 of each respective leaflet. For example, the leaflets 30a, 30b, and 30c can be selected to have deflections, about the first axis 49 of each respective leaflet, under gravity, that vary from one another by less than about 0.125 inches. In certain embodiments, the leaflets 30a, 30b, and 30c are selected to have similar deflections about the second axis 51 of each respective leaflet. For example, the leaflets 30a, 30b, and 30c can be selected to have deflections, about the second axis 51 of each respective leaflet under gravity, that vary from one another by less than about 0.125 inches.

Additionally or alternatively, leaflet moistening can be used to reduce the likelihood of delamination of the leaflets 30a, 30b, 30c when arranged to coapt with one another to form a coaptation region of about 15 percent to about 35 percent of the overall height of each of the plurality of leaflets. For example, each leaflet 30a, 30b, 30c of the leaflet assembly 16 can be moistened before and after being assembled into the leaflet assembly 16. Additionally or alternatively, each leaflet 30a, 30b, 30c of the leaflet assembly 16 can be moistened before and after the leaflet assembly 16 is coupled to the stent 18.

The leaflets 30a, 30b, 30c can be moistened, for example, by exposing each leaflet to a moistening solution such as saline. For example, the leaflets 30a, 30b, 30c can each be stored in the moistening solution. Additionally or alternatively, the leaflet assembly 16 including the leaflets 30a, 30b, 30c can be stored in a moistening solution with the leaflet assembly 16 in a substantially open position (e.g., the leaflets 30a, 30b, and 30c slightly separated from one another to allow fluid to flow therethrough).

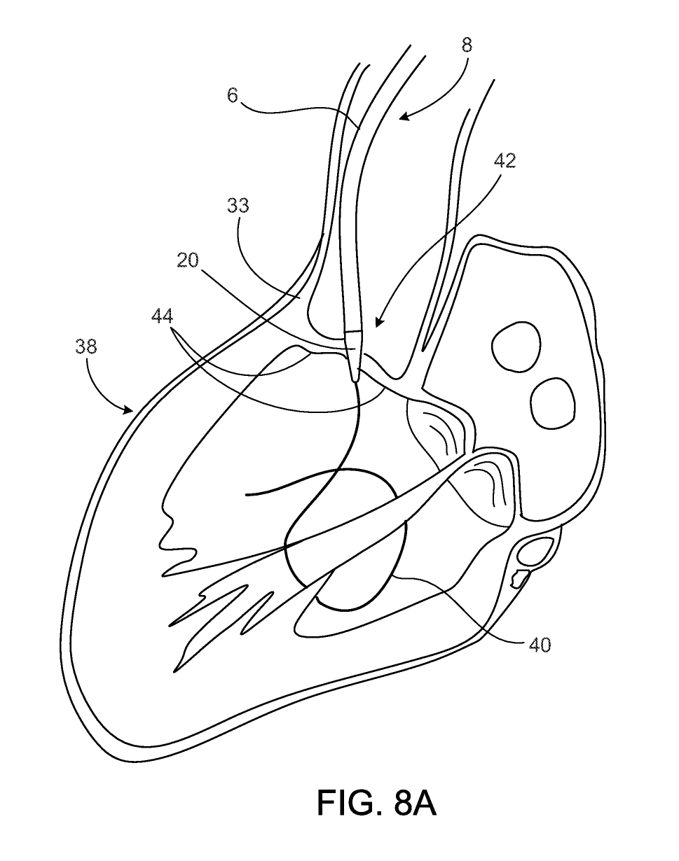

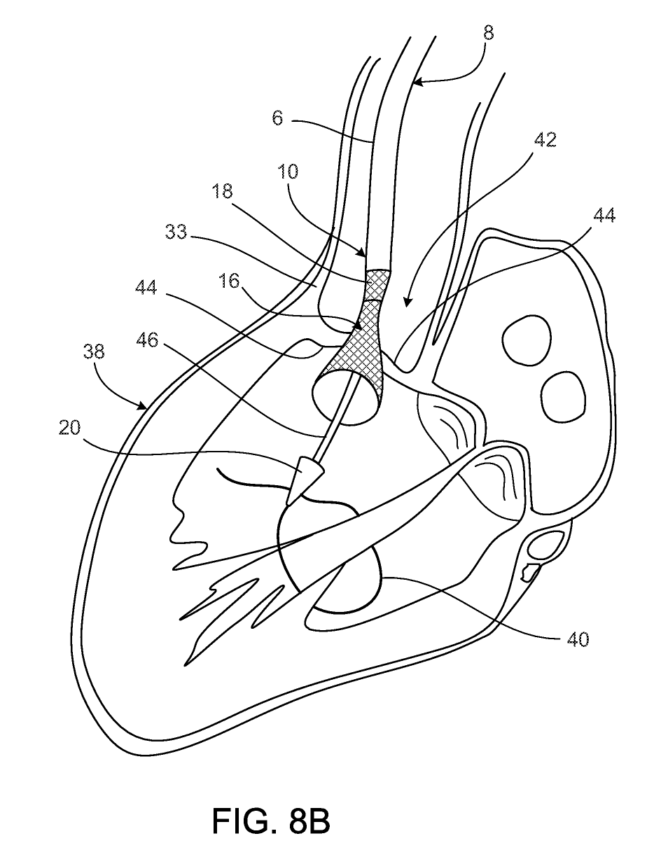

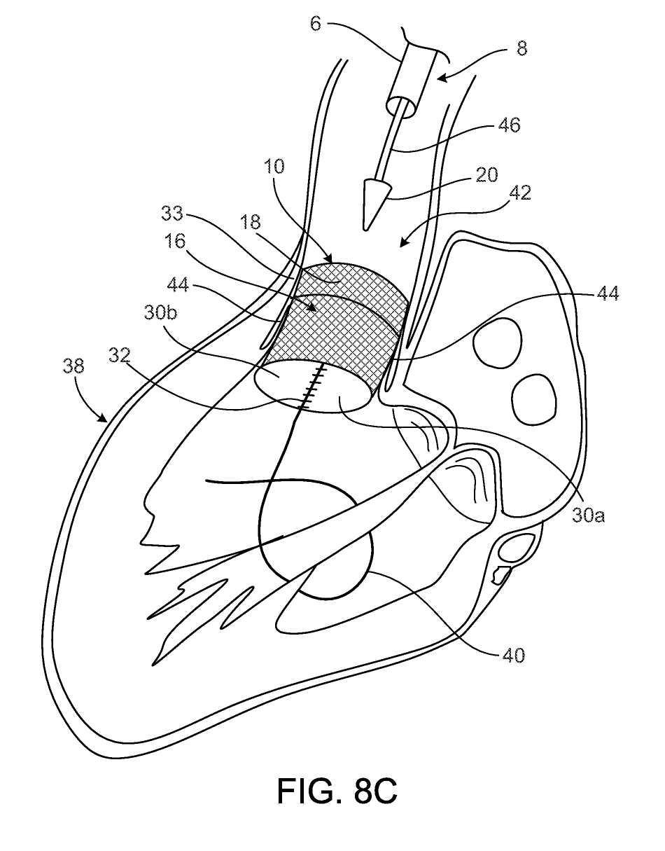

Referring now to FIGS. 1 and 8A-C, the delivery system 1 can be used for intraluminal delivery of the replacement valve 10 to an aortic valve 42 of a mammalian heart 38, where the replacement valve 10 can be deployed without the need for excising the native leaflets 44 of the aortic valve 42. The distal portion 8 of the delivery system 1 is moved over a guidewire 40 (e.g., by manipulation of the control handle 2) until the nosecone 20 moves past the native leaflets 44. With the distal portion 8 of the delivery system 1 in place, the external sheath 6 is retracted (e.g., by manipulation of the control handle 2) to release the replacement valve 10. The released replacement valve 10 can expand radially under the self-expanding force of the stent 18. Additionally or alternatively, the released replacement valve 10 can expand radially under the force of the actuation elements 12, which can also be manipulated by the control handle 2.

The force of the fully expanded stent 18 secures the replacement valve 10 to the wall of the aortic valve 42 and pins the native leaflets 44 to an aortic wall 33. With the native leaflets 44 pinned in this position, the leaflet assembly 16 opens and closes in response to the pulsatile flow of blood through the heart 38 and, in this way, acts to replace the aortic valve 42. After the replacement valve 10 has been fully deployed in the aortic valve 42, the nosecone 20 can be retracted proximally through the valve by an inner tube 46 and the distal portion 8 of the delivery system 1 can be retracted proximally along the guidewire 40 until the delivery system 1 is removed from the recipient of the replacement valve 10.

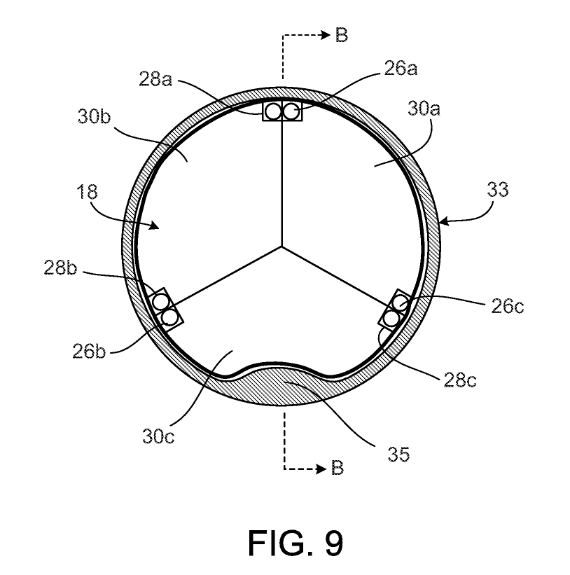

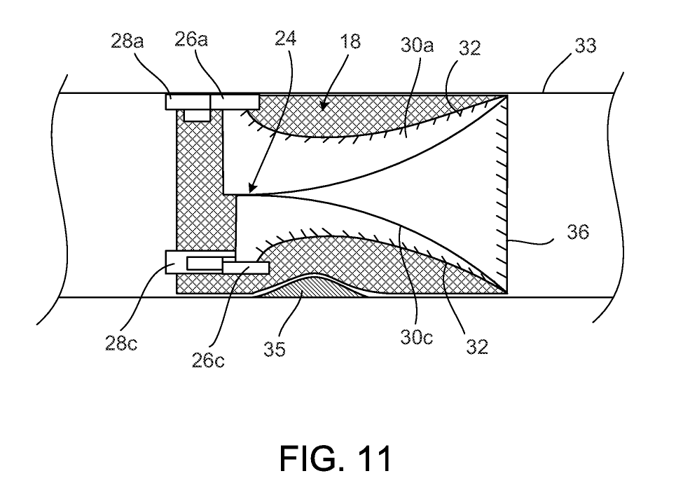

Referring now to FIGS. 9 to 11, the aortic wall 33 is itself naturally non-circular and can be the site of deposit build-up. As described above, since the replacement valve 10 is intraluminally delivered to the aortic valve 42, the implantation site cannot be prepared (e.g., made to be substantially round) before the replacement valve 10 is implanted. Rather, the stent 18 of the replacement valve 10 is flexible to conform to the non-circular aortic wall 33 such that the stent 18 can define a substantially non-uniform cross-sectional area along its length. For example, as shown in FIGS. 9 to 11, the replacement valve 10 can be implanted such that at least a portion of the stent 18 conforms to accommodate a deposit 35 on the aortic wall 33.

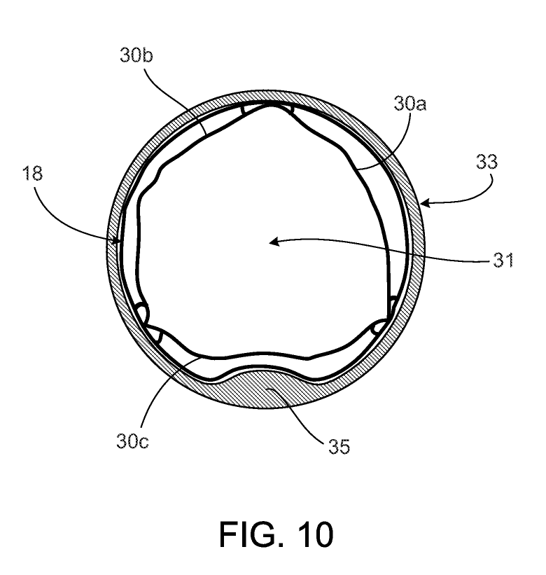

Since the leaflet assembly 16 is coupled to the stent 18, the non-uniform flexing of the stent 18 in response to the deposit 35 can change the relative orientation of the leaflets 30a, 30b, 30c. For example, the leaflets 30a, 30b, 30c may coapt differently as compared to the coaptation exhibited when the stent 18 is in the fully expanded, unstressed state. The leaflets 30a, 30b, 30c are sized such that the coaptation region 24 ensures full closure of the leaflet assembly 16 when the stent 18 flexes to accommodate the deposit 35, provided that the deposit 35 is not of such a size and shape to cause one or more of the leaflets 30a, 30b, 30c to contact the stent 18 when the leaflet assembly 16 is in the open position (e.g., the open position shown in FIG. 10). That is, the possibility of one or more of the leaflets 30a, 30b, 30c coming into contact with the stent 18 can provide an upper boundary condition on the size of the leaflet coaptation region 24.

A number of embodiments of the invention have been described. Nevertheless, it will be understood that various modifications may be made without departing from the spirit and scope of the invention. For example, the replacement valve can include one or more of a plurality of anchors for penetrating native tissue to secure the replacement valve in place. Accordingly, other embodiments are within the scope of the following claims.

What is claimed is:

1. A prosthetic heart valve comprising:
a stent radially expandable from a first size for intraluminal delivery through a body passageway to a second size for implantation of the prosthetic heart valve in the body passageway; and
a leaflet assembly coupled to the expandable stent, the leaflet assembly comprising a plurality of leaflets movable between an open position permitting flow past the expanded stent and a closed position substantially restricting flow past the expanded stent, each of the plurality of leaflets coaptable with each of the other leaflets in the closed position to define a coaptation region of about 15 percent to about 35 percent of the overall height of each of the plurality of leaflets.

2. The prosthetic heart valve of claim 1 wherein at least a portion of each leaflet of the plurality of leaflets is sutured to at least a portion of each of the other leaflets of the plurality of leaflets.

3. The prosthetic heart valve of claim 1 wherein each leaflet of each of the plurality of leaflets has a first end portion, second end portion, and a belly portion extending therebetween, the first end portion of each leaflet secured to the stent and the second end portion of each leaflet movable relative to the respective second end portions of each of the other leaflets as the leaflets move from the closed position to the open position.

4. The prosthetic heart valve of claim 3 wherein each leaflet is sized such that the belly portion of each respective leaflet is spaced from the stent.

5. The prosthetic heart valve of claim 1 wherein each leaflet is deflectable about a first axis defined by the leaflet and the respective deflections of the plurality of leaflets about the respective first axes under gravity varies from one another by less than about 0.125 inches.

6. The prosthetic heart valve of claim 5 wherein each leaflet is deflectable about a second axis defined by the leaflet and the respective deflections of the plurality of leaflets about the respective second axes under gravity varies from one another by less than about 0.125 inches.

7. The prosthetic heart valve of claim 1 wherein the coaptation region is defined by engagement of three leaflets of the plurality of leaflets.

8. The prosthetic heart valve of claim 1 wherein the plurality of leaflets comprise biological tissue.

9. The prosthetic heart valve of claim 8 wherein the biological tissue is one or more of the following: bovine pericardium, equine pericardium, and porcine pericardium.

10. The prosthetic heart valve of claim 1 wherein at least a portion of the stent is deformable to define a non-circular cross-section when implanted in the body passageway.

11. A method of manufacturing a prosthetic heart valve comprising:
forming a leaflet assembly, the forming comprising engaging a plurality of leaflets with one another; and
securing the leaflet assembly to a radially expandable stent such that the plurality of leaflets are movable between an open position permitting flow past the expanded stent and a closed position substantially restricting flow past the expanded stent, each of the plurality of leaflets coaptable with each of the other leaflets in the closed position to define a coaptation region of about 15 percent to about 35 percent of the overall height of each of the plurality of leaflets.

12. The method of claim 11 wherein forming the leaflet assembly further comprises cutting each of the plurality of leaflets from a sheet of biological tissue.

13. The method of claim 12 wherein cutting each leaflet comprises pressing a steel die on the sheet of biological tissue.

14. The method of claim 12 wherein the biological tissue is one or more of the following: bovine pericardium, equine pericardium, and porcine pericardium.

15. The method of claim 11 further comprising moistening each leaflet of the leaflet assembly.

16. The method of claim 15 wherein moistening each leaflets of the leaflet assembly comprises exposing each leaflet to a moistening solution before and after forming the leaflet assembly.

17. The method of claim 15 wherein moistening each leaflet of the leaflet assembly comprises exposing each leaflet to a moistening solution before and after securing the leaflet subassembly to the radially expandable stent.

18. The method of claim 15 wherein moistening each leaflet of the leaflet assembly comprises storing each leaflet in a moistening solution.

19. The method of claim 11 further comprising selecting the plurality of leaflets, wherein each leaflet defines a first axis and selecting the plurality of leaflets comprise selecting leaflets that deflect about each of their respective first axes under gravity by an amount that varies from one another by less than about 0.125 inches.

20. The method of claim 19 wherein each leaflet defines a second axis and selecting the plurality of leaflets further comprises selecting the leaflets that deflect about each of their respective second axes under gravity by an amount that varies from one another by less than about 0.125 inches.
